(19) United States
(12) Patent Application Publication
(10) Pub. No.: US 2004/0133124 A1
(43) Pub. Date: Jul. 8, 2004

(54) Flexible biopsy needle

(75) Inventors: Brian L. Bates, Bloomington, IN (US); Robert S. Childress, Solsberry, IN (US)

Correspondence Address: Brinks Hofer Gilson & Lione, P.O. Box 10395, Chicago, IL, 60610, US

(73) Assignee: Cook Incorporated.

(21) Appl. No.: 10/337,109

(22) Filed: Jan. 6, 2003

(51) Int. Cl. A61B 10/00

(52) U.S. Cl. 600/564

(57) ABSTRACT

A flexible biopsy needle comprising a cannula, a tissue penetrating stylet slidably disposed within the cannula, and a handle mechanism to advance the cannula over the stylet that can be bent and still retain the ability for the cannula to move smoothly and freely over the stylet.

### BACKGROUND OF THE INVENTION

1. Field of the Invention

The present invention relates to a surgical instrument and, particularly, to a flexible instrument for biopsy sampling of tissue.

2. Background of Related Art

Biopsy is the removal and study of body tissue for medical diagnosis. Typically, physicians obtain biopsy samples in order to detect abnormalities such as cancer and determine the extent to which cancerous tissue has spread. They use various biopsy instruments to acquire tissue samples from different areas of the body.

Typically, current biopsy instruments comprise a two-part needle assembly, or a stylet and cannula, operated by a spring-loaded handle of the type disclosed in U.S. Pat. No. 5,538,010, the disclosure of which is incorporated herein by reference. Another conventional biopsy instrument is the Cook, Incorporated QUICK-CORE™ Biopsy Needle 5′ shown in FIGS. 1-1D. The inner part or stylet 30′ of the needle 5′ has a tissue collecting or specimen notch 50 formed near a stylet distal end. The outer part or cannula 10′ has a point 19 on the cannula distal end 14 and encloses the stylet 30′. The cannula 10′ and stylet 30′ are arranged so that the cannula point 19 advances over the style in order to cover the specimen notch 50. In use, this forward movement of the cannula 10 cuts out a specimen of the prolapsed tissue, which specimen becomes retained in the specimen notch 50 of the stylet 30′. The QUICK-CORE™ Biopsy Needle 5′ may then be withdrawn and the tissue sample recovered from the stylet 30′. The spring-loaded handle 60 advances the cannula 10′ over the stylet 30′ very quickly in order to prevent the prolapsed tissue in the specimen notch 50 from being displaced as the cannula 10′ advances over the stylet 30′.

This system works very well for a variety of biopsy procedures. It is, however, limited to a “straight shot” procedure in which the needle is inserted percutaneously directly to a target lesion, and the ability of the needle to negotiate through the vasculature of a patient is limited. In many situations, a physician may desire to insert the biopsy needle through an outer or guiding catheter to biopsy a target lesion without having to proceed through healthy tissue. For example, a physician may desire to obtain liver histology samples via the jugular vein. Under this approach, the physician uses the QUICK-CORE™ Biopsy Needle 5′ in conjunction with a biopsy set 70 as shown in FIGS. 2-2A. Preferably, the biopsy set 70 includes: a stiffening cannula 71 to provide directional control and backup support of the QUICK-CORE™ ™ Biopsy Needle, a hemostasis valve 72 and adapter 73, such as the Cook Incorporated CHECK-FLO® Valve Adapter, to prevent back bleeding and permit contrast injection for visualization of the target area prior to biopsy, an introduction sheath 74; a straight guiding catheter 75; and a curved guiding catheter 76. More preferably, the stiffening cannula 71 is a 14 gauge stainless steel cannula having an extra thin wall and a length of about 50.5 cm; the introduction sheath 74 is 7.0 French radiopaque FEP sheath having a length of 49 cm; the straight guiding catheter 75 is a 5.0 French Teflon catheter having a length of about 62 cm; and the curved guiding catheter 76 is a 5.0 French radiopaque braided nylon catheter having a length of about 80 cm, such as the TORCON NB® ADVANTAGE catheter, for example.

First, the physician punctures the right internal jugular vein with an access needle. Next, a wire guide is inserted through the needle and into the vein and the access needle is removed. An outer or guiding catheter, such as the 5.0 French TORCON NB® ADVANTAGE multipurpose catheter, for example, is introduced over the wire guide and manipulated through the vasculature of the patient to the right hepatic vein. Once the wire guide is seated in the right hepatic vein, the guiding catheter is removed. Depending on the location of the target area, the physician may alternatively select the straight guiding catheter 75 in place of the curved guiding catheter 76. Next, the preassembled liver access set 77 comprising the introduction sheath 74, the stiffening cannula 71 and the CHECK-FLO® Valve Adapter 72, 73 is advanced over the wire guide and into the right hepatic vein as shown in FIG. 3. They physician my inject contrast through the side-arm fitting of the CHECK-FLO® Valve Adapter to verify the location within the right hepatic vein. The liver access set 77 is directed anteriorly and gentle forward pressure is applied to “tent” the walls of the hepatic vein adjacent to the biopsy site as shown in FIG. 3. At this point, the QUICK-CORE™ Biopsy Needle 5′ is advanced through the liver access set until the QUICK-CORE™ Needle tip 39 is positioned at the distal end of the access assembly 77 as shown in FIG. 4. Preferably, the QUICK-CORE™ Biopsy Needle 5′ includes an etch mark 15 on the proximal portion of the cannula that indicates the QUICK-CORE™ Needle tip is at the distal end of the access assembly. Next, the QUICK-CORE™ Biopsy Needle is advanced out of the assembly's tip and into the liver tissue as shown in FIGS. 5-5A. The physician maintains the position of the needle and advances the stylet to expose the specimen notch 50 within the area to be biopsied (as shown in FIG. 5A) and fires the cutting cannula to capture the prolapsed tissue within the specimen notch 50. Although the QUICK-CORE™ Biopsy Needle has enjoyed some success with this procedure, the bends and curves encountered as the biopsy needle is manipulated through the vasculature of a patient can cause the stylet 30 and cannula 10 to bind and prevent the cannula 10 from advancing over the stylet 30.

In light of the foregoing, there exists a need for an inexpensive biopsy tissue-sampling device of a simple design that effectively negotiates the vasculature of a patient and still retains the ability for the cannula outer portion to move smoothly and freely over the inner stylet potion.

### BRIEF SUMMARY OF THE INVENTION

The foregoing problems are solved and a technical advantage is achieved by the present invention, which is a surgical instrument for excising a targeted tissue mass to be biopsied from adjacent bodily tissue in a surgical patient. More specifically, the present invention is a flexible biopsy needle.

In one embodiment, the invention is a surgical cutting instrument comprising a cannula having a cannula proximal end, a cannula distal end, a cannula hollow passageway positioned longitudinally therebetween and a cannula outer surface including at least one cannula notch positioned between said cannula proximal end and cannula distal end; a stylet comprising a stylet proximal end, a stylet distal end, a recessed channel positioned proximal said distal end, said recessed channel extending laterally across said hollow passageway and comprising oppositely facing edges, said stylet further comprising an outer surface including at least one stylet notch positioned between said stylet proximal end and proximal said recessed channel; said stylet coaxially and slidably positioned within said cannula hollow passageway such that said at least one cannula notch overlaps said at least one stylet notch; and a handle mechanism connected to said cannula and said stylet, said handle mechanism allowing relative movement between said cannula and said stylet such that said stylet may be extended between a first position, wherein said recessed channel is retracted within said cannula distal end, and a second position, wherein said recessed channel is axially extended beyond said cannula distal end.

### BRIEF DESCRIPTION OF SEVERAL VIEWS OF THE DRAWINGS

FIG. 1 illustrates a prior art biopsy needle.

FIG. 1A is a plan view of a prior art biopsy needle of FIG. 1 with an enlarged view of the distal portion.

FIG. 1B is a partial, side view of a distal portion of the biopsy needle of FIG. 1A.

FIG. 1C is a plan view of another version of the biopsy needle of FIG. 1 with an enlarged view of the distal portion.

FIG. 1D is a partial, side view of the distal portion of the biopsy needle of FIG. 1C.

FIG. 2 illustrates a prior art liver access set comprising a hemostasis valve and adapter, a stiffening cannula, an introduction sheath, a straight guiding catheter and a curved guiding catheter.

FIG. 2A illustrates a liver access of FIG. 2 set comprising a hemostasis valve and adapter, a stiffening cannula, and an introduction sheath assembled with the biopsy needle of FIG. 1.

FIG. 3 illustrates a liver access set of FIG. 2A positioned in the right hepatic vein such that the walls of the hepatic vein protrude adjacent to the biopsy site.

FIG. 4 illustrates the biopsy needle of FIG. 1 advanced through the liver access set of FIG. 2A such that the biopsy needle tip is positioned at the distal end of the access assembly.

FIG. 5 illustrates the biopsy needle of FIG. 1 advanced out of the tip of the liver access of FIG. 2A and into the target tissue.

FIG. 5A illustrates the biopsy needle of FIG. 5 with the stylet advanced to expose the specimen notch within the area to be biopsied.

FIG. 6 depicts a plan view of one preferred embodiment of the flexible surgical cutting instrument of the present invention.

FIG. 7 depicts a partial, side view of the flexible surgical cutting instrument of FIG. 6 in a straight configuration showing the cannula retracted and the tissue notch exposed.

FIG. 8 depicts a partial, side view of the stylet of the flexible surgical cutting instrument of FIG. 6 in a straight configuration.

FIG. 9 depicts a partial, side view of the cannula of the flexible surgical cutting instrument of FIG. 6 in a straight configuration.

FIG. 10 depicts the flexible surgical cutting instrument of FIG. 7 in a curved or bent configuration illustrating the cannula advanced over the specimen notch.

FIG. 11 depicts a partial, side view of the stylet of FIG. 8 in a curved, or bent, configuration.

FIG. 12 depicts a partial side view of the cannula of FIG. 9 in a curved configuration.

FIG. 13 depicts a partial, side view of another preferred embodiment of the flexible surgical cutting instrument of the invention in a straight configuration showing the cannula retracted and the tissue notch exposed.

FIG. 14 depicts a partial, side view of the stylet of the flexible surgical cutting instrument of FIG. 13 in a straight configuration.

FIG. 15 depicts a partial, side view of the cannula of the flexible surgical cutting instrument of FIG. 13 in a straight configuration.

FIG. 16 depicts the flexible surgical cutting instrument of FIG. 13 in a curved, or bent, configuration illustrating the cannula advanced over the specimen notch.

FIG. 17 depicts a partial, side view of the stylet of FIG. 14 in a curved configuration.

FIG. 18 depicts a partial, side view of the cannula of FIG. 15 in a curved configuration.

### DETAILED DESCRIPTION OF THE INVENTION

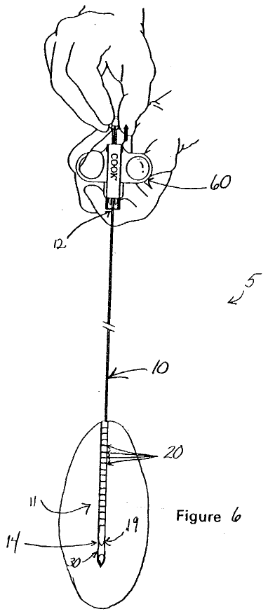

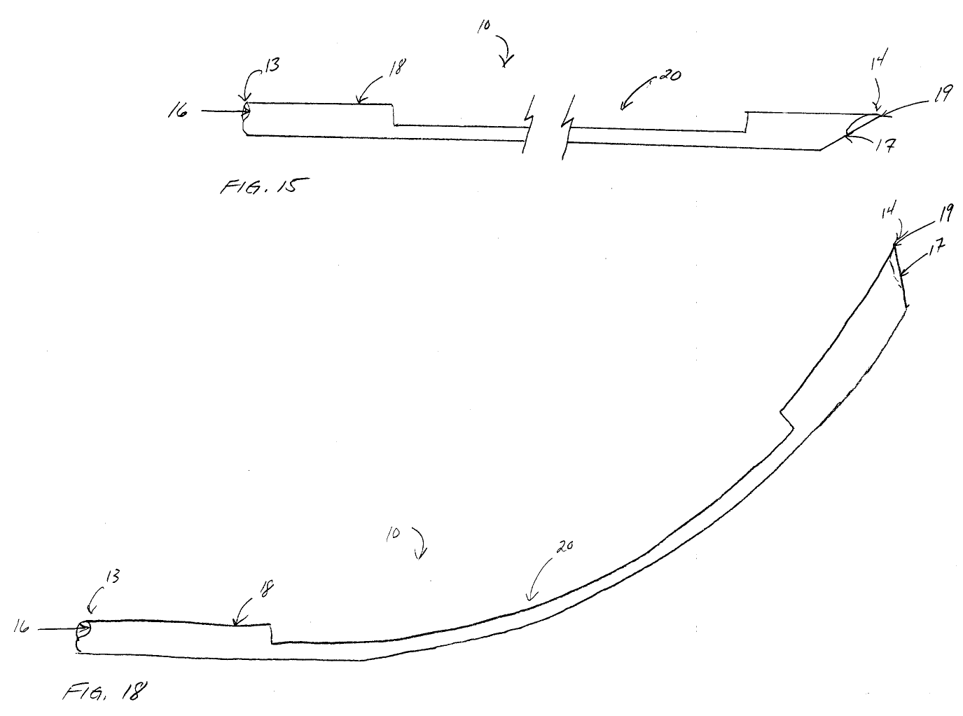

Referring to FIGS. 6-18, there are illustrated various embodiments of the flexible surgical cutting instrument 5 for excising a targeted tissue mass to be biopsied. The biopsy surgical instrument comprises an elongated tube or cannula 10, a tissue penetrating stylet 30 and a handle mechanism 60 connected to the cannula 10 and the stylet 30. The handle mechanism 60 permits relative movement between the cannula 10 and the stylet 30 and advances the cannula 10 over the stylet 30. Preferably, the handle mechanism 60 is a spring-loaded handle, such as, by way of a non-limiting example, of the type described in U.S. Pat. No. 5,538,010, the disclosure of which is incorporated by reference.

The cannula 10 includes proximal and distal ends 12, 14, respectively, a hollow passageway 16 positioned longitudinally therebetween, proximal portion, a distal portion 13 and a cannula diameter. As used herein the term “proximal portion” refers to a portion proximal a midpoint and the term “distal portion” refers to a portion distal to the midpoint. As a non-limiting example, the cannula 10 may be a stainless steel tube having a diameter of about 0.330 inch and a wall thickness of about 0.010 inch. The cannula 10 may also be constructed from any other suitable material including, but not limited to, metals, metal alloys such as nickel titanium alloys known to be shape-memory metals which are sold and manufactured under the trademark “NITINOL,” and rigid or semi-rigid plastics. It is also anticipated that new materials, as they are developed, will be useful.

As shown in FIGS. 6-7, 9-10, 12-13, 15-16, and 18, the cannula 10 further comprises a wall 18 having at least one cannula notch 20 positioned proximal the cannula distal end 14. Preferably, the at least one cannula notch 20 is positioned along the cannula wall on the cannula distal portion 13.

As used herein, the term “notch” is defined as a location where material has been removed or a location that has been formed without material. The distance along which the at least one cannula notch 20 is positioned controls the radius of curvature of cannula 10 that results and can be varied. The at least one cannula notch 20 may be positioned along the cannula wall 18 over a distance between the cannula proximal end 12 and the cannula distal end 14. Preferably, the at least one cannula notch 20 is positioned along the cannula wall 18 proximal the cannula distal end 14 over a distance from about 5 cm to about 10 cm. Most preferably, the at least one cannula notch is positioned near the distal end 14 over a distance from about 5 cm to about 10 cm.

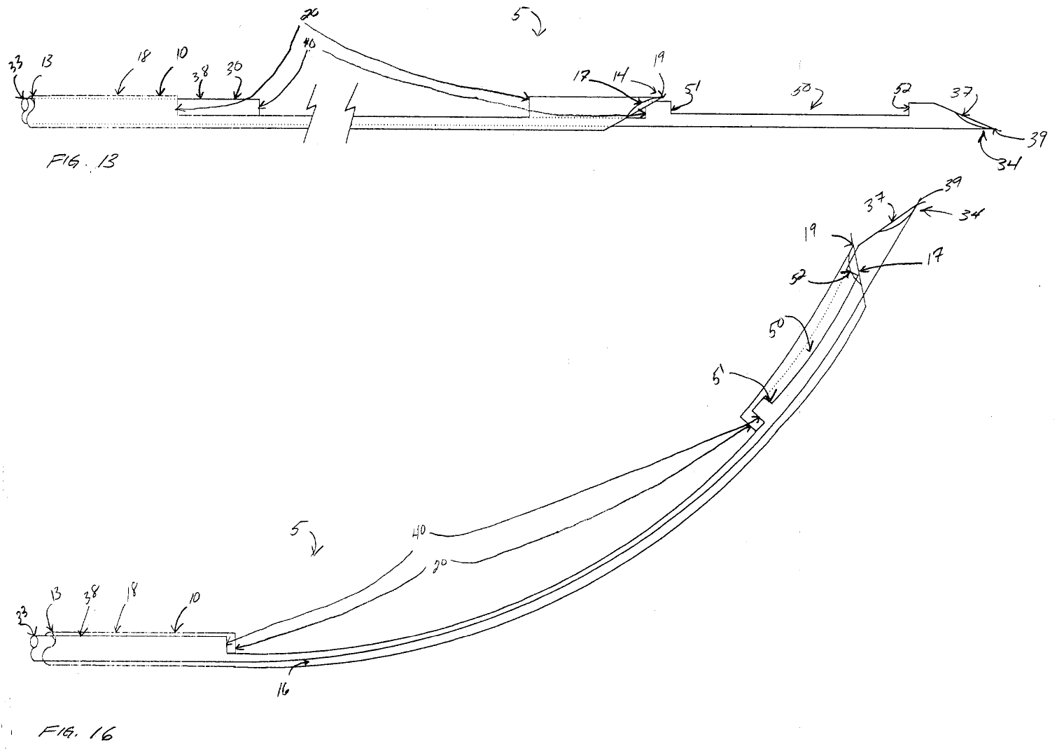

In one preferred embodiment, shown in FIGS. 7, 9-10 and 12, the cannula wall 18 comprises a plurality of cannula notches 20. In another preferred embodiment shown in FIGS. 13, 15-16 and 18, a desired distance of the cannula wall 16 is removed to form a single notch 20. Preferably, the at least one cannula notch 20 reduces the periphery of the cannula wall 18 by approximaltely one half. More preferably, the at least one cannula notch 20 reduces the periphery of the cannula wall 18 by one half or more.

As shown in FIGS. 6-7, 9-10, 12-13, 15-16, and 18, the distal end 14 of cannula 10 defines a shearing end comprising a shearing edge 17 which terminates in a forward shearing point 19. Preferably, shearing end comprises a tapered shearing edge. More preferably, the tapered shearing edge comprises an annular, beveled edge about the distal end 14 of cannula 10. Most preferably, the beveled edge comprises an inner surface and an outer surface. The inner surface extends axially beyond the outer surface to preclude the prolapsed tissue from catching as the cannula 10 and the stylet 30 are negotiated to a target lesion.

A tissue penetrating stylet 30 is positioned within and mounted for axial movement through the cannula hollow passageway 18 as shown in FIGS. 6-7, 10, 13 and 16. The stylet 30 comprises a proximal end, a distal end 34, a proximal portion, a distal portion 33 and a stylet diameter. As a non-limiting example, the stylet 30 may be stainless steel having a diameter of about 0.300 inch. The stylet may also be constructed from any other suitable material including, but not limited to, metals, metal alloys such as nickel titanium alloys known to be shape-memory metals which are sold and manufactured under the trademark “NITINOL,” and rigid or semi-rigid plastics. It is also anticipated that new materials, as they are developed, will be useful.

As shown in FIGS. 7, 8, 10-11, 13-14 and 16-17, the stylet 30 further comprises a surface 38 having at least one stylet notch 40. The stylet surface 38 comprises at least one stylet notch 40 positioned proximal the stylet distal end 34. Preferably, the at least one stylet notch 40 is positioned along the stylet surface on the stylet distal portion 33.

The distance along which the at least one stylet notch 40 is positioned determines the radius of curvature of stylet 30 that results and can be varied. The at least one stylet notch 40 may be positioned along the stylet surface 38 over a distance from between the stylet proximal end and the stylet distal end 34. Preferably, the at least on stylet notch 40 is positioned along the stylet surface 38 over a distance from about 5 cm to about 10 cm.

In one preferred embodiment, shown in FIGS. 7-8 and 10-11, the stylet surface 38 comprises a plurality of stylet notches 40. In this embodiment, a series of notches is formed in the stylet surface 36 over a desired distance. In another preferred embodiment, a desired distance of material is removed from the stylet surface 38 to form a single stylet notch 40 as shown in FIGS. 13-14 and 16-17. Preferably, the at least one stylet notch 40 reduces the periphery of stylet surface 38 by approximately one half. More preferably, the at least one stylet notch 40 reduces the periphery of stylet surface 38 by one half or more.

As shown in FIGS. 7-8, 10-11, 13-14 and 16-17, the stylet 30 further comprises a recessed channel or specimen notch 50 positioned proximal the stylet distal end 34. Preferably, the specimen notch 50 is located in the stylet distal portion 33. Preferably, the at least one stylet notch 40 is positioned along the stylet surface 38 proximal the specimen notch 50. The specimen notch 50 comprises oppositely facing edges 51, 52. Preferably, the depth and expanse of specimen notch 50 are sized to hold a tissue specimen of adequate size for conventional examination. Preferably, the specimen notch 50 has a length of up to about 3 to about 3.5 cm and more preferably, the recessed channel 50 has a length of up to about 1 cm to about 1.5 cm. Preferably, the specimen notch 50 has a depth equal to about one half the stylet diameter.

As shown in FIGS. 7-8, 10-11, 13-14 and 16-17, the stylet distal end 34 defines a piercing end. Preferably, the piercing end comprises a tapered face having a cutting edge 37 terminating in a forward piercing point 39.

In another embodiment, stylet 30 may further comprise a stylet hollow passageway positioned longitudinally between stylet proximal end and stylet distal end 34. As a non-limiting example of this embodiment, stylet surface 38 may have a thickness of about 0.010 inch. Preferably, specimen notch 50 extends laterally across stylet hollow passageway 38 on the stylet distal portion 33.

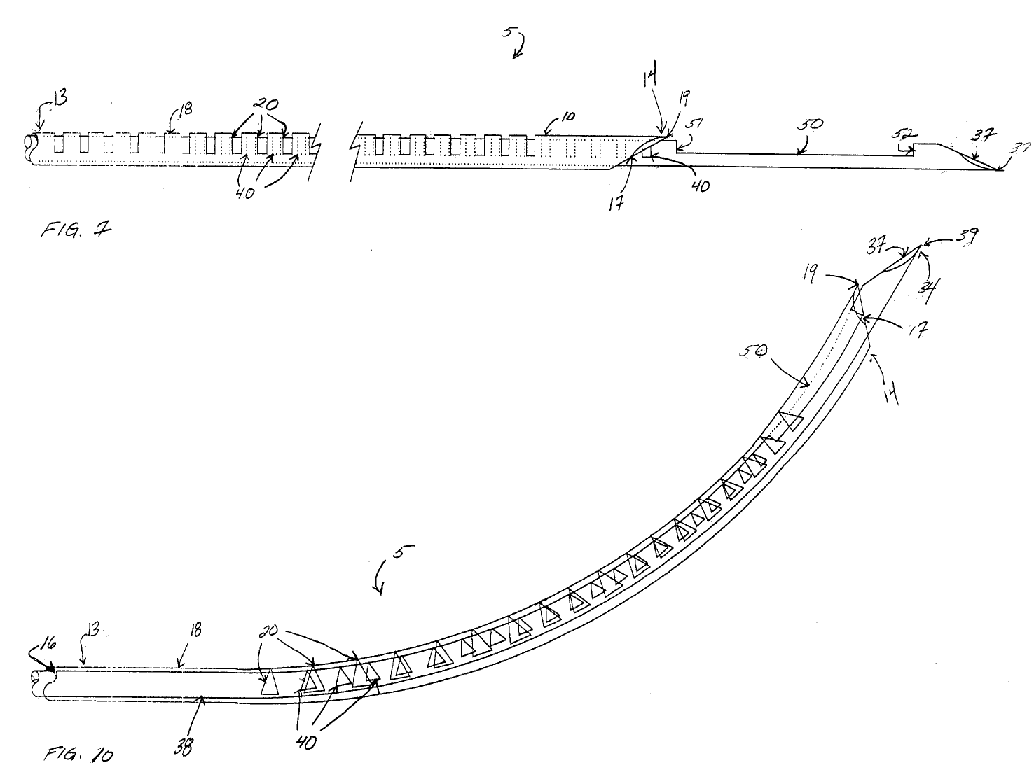

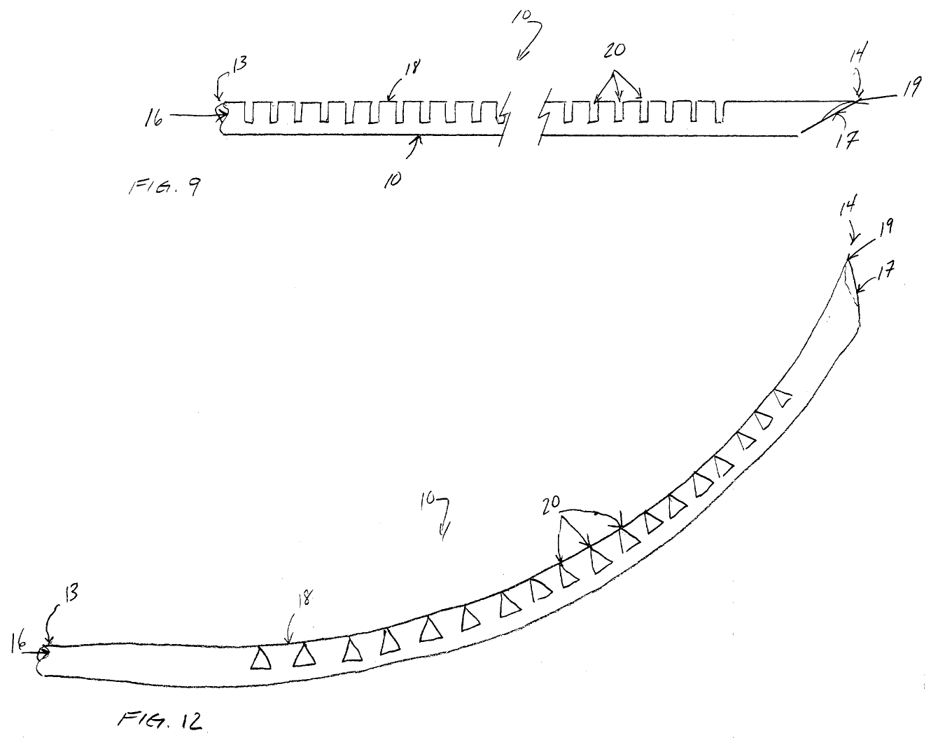

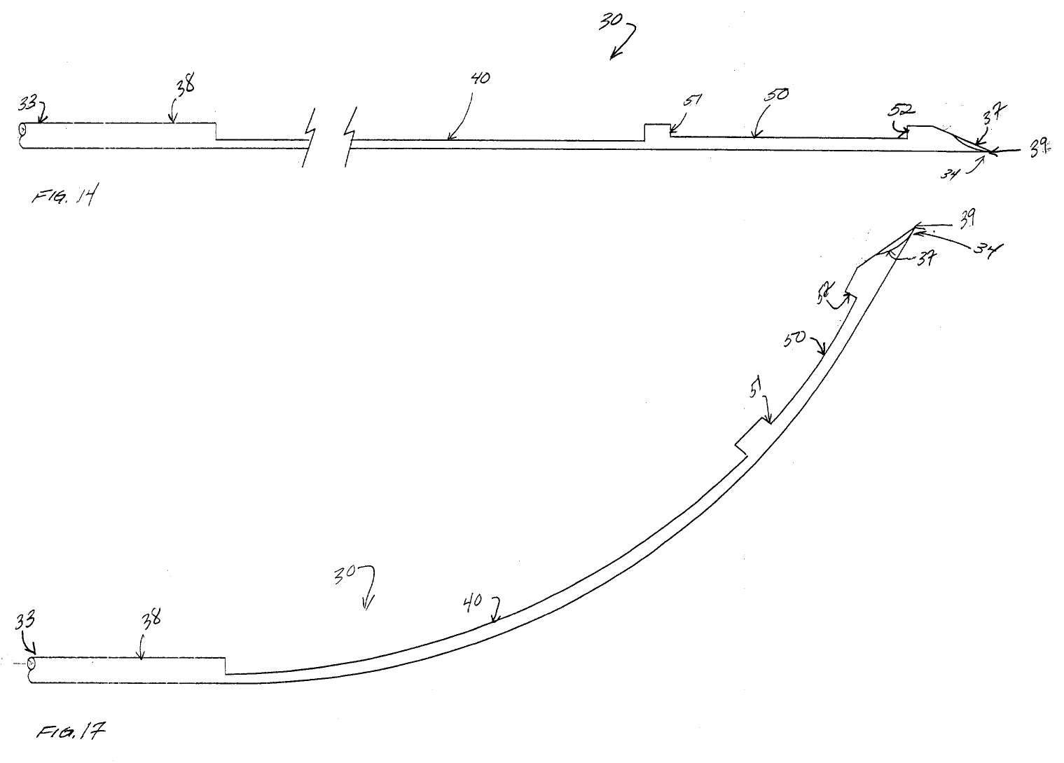

FIGS. 8 and 14 show the stylet in the straight or unbent configuration and FIGS. 9 and 15 shows the cannula 10 in the straight or unbent configuration. In the straight or unbent configuration, the stylet and cannula notches 40, 20, respectively, define an opening along the stylet and cannula walls, 38, 18 respectively. As the stylet 30 and cannula 20 are flexed to a curved or bent configuration, the stylet notch 40 and the cannula notch 20 will, at the limit, close on the inside of the bend as shown in FIGS. 11 and 12. The at least one cannula and the at least one stylet notches 20, 40 may comprise a variety of shapes as long as the selected shape has an opening along the cannula wall 18 and stylet surface 38 including, but not limited to: grooves, slots, u-shapes, teardrops, semicircles etc. In addition, the cannula and stylet notches 20, 40 may also vary in size. The distance along which the at least one cannula and the at least one stylet notches 20, 40 are positioned controls the radius of curvature that results.

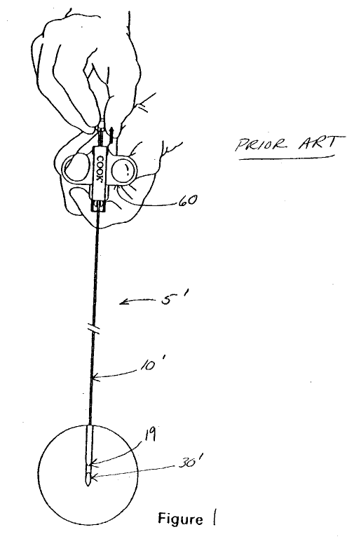

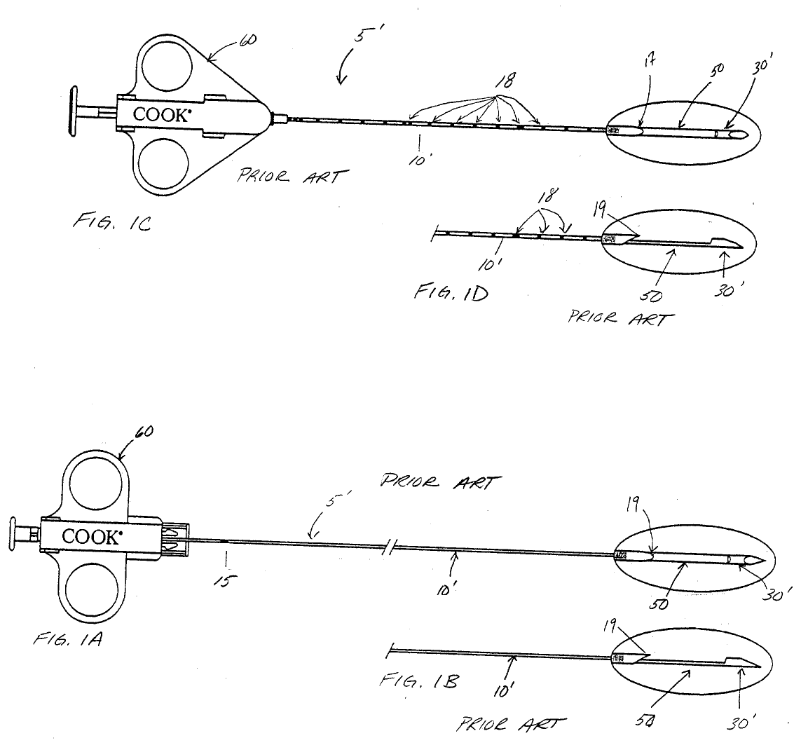

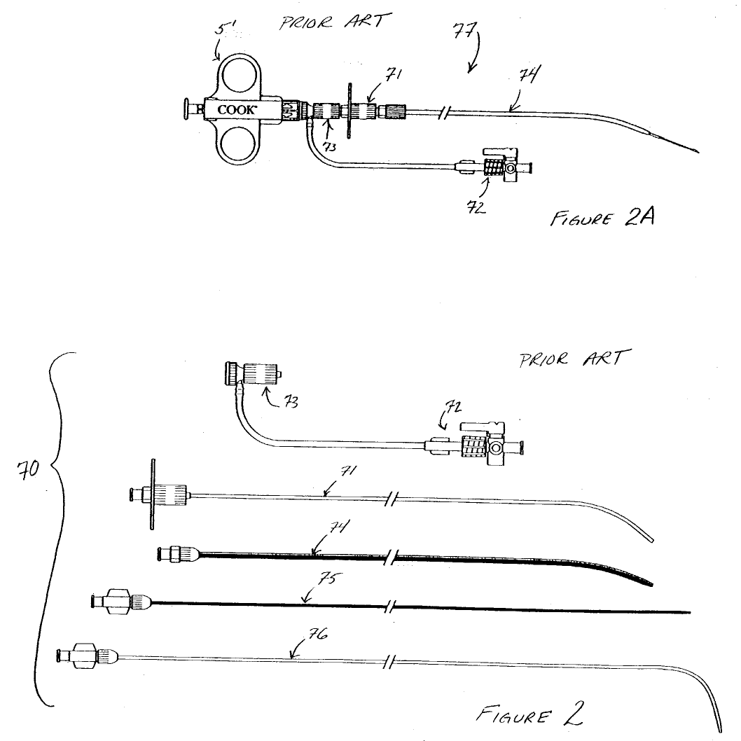

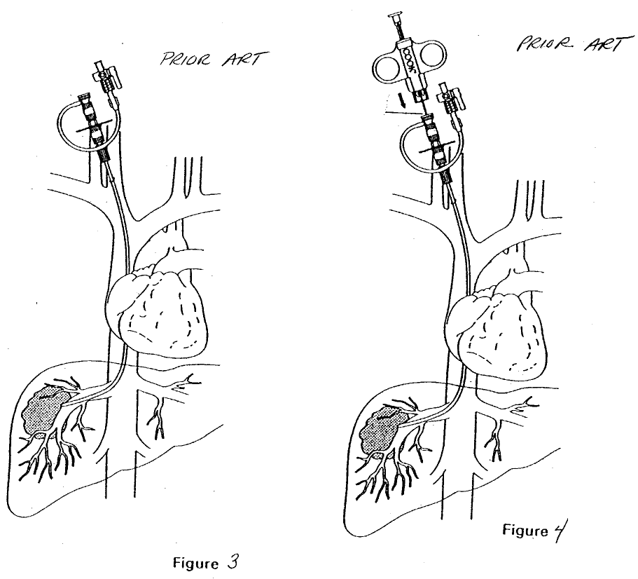

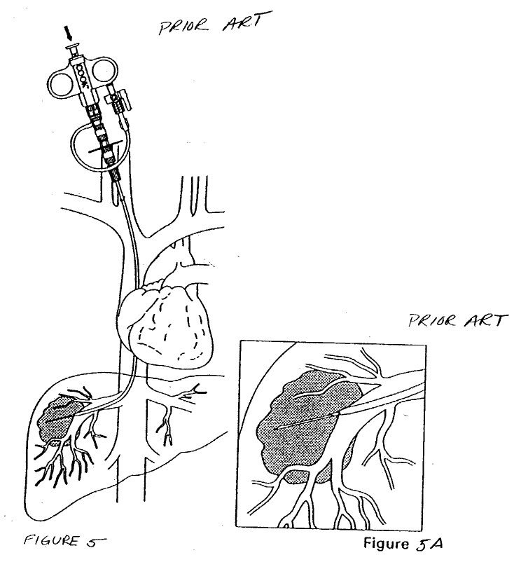

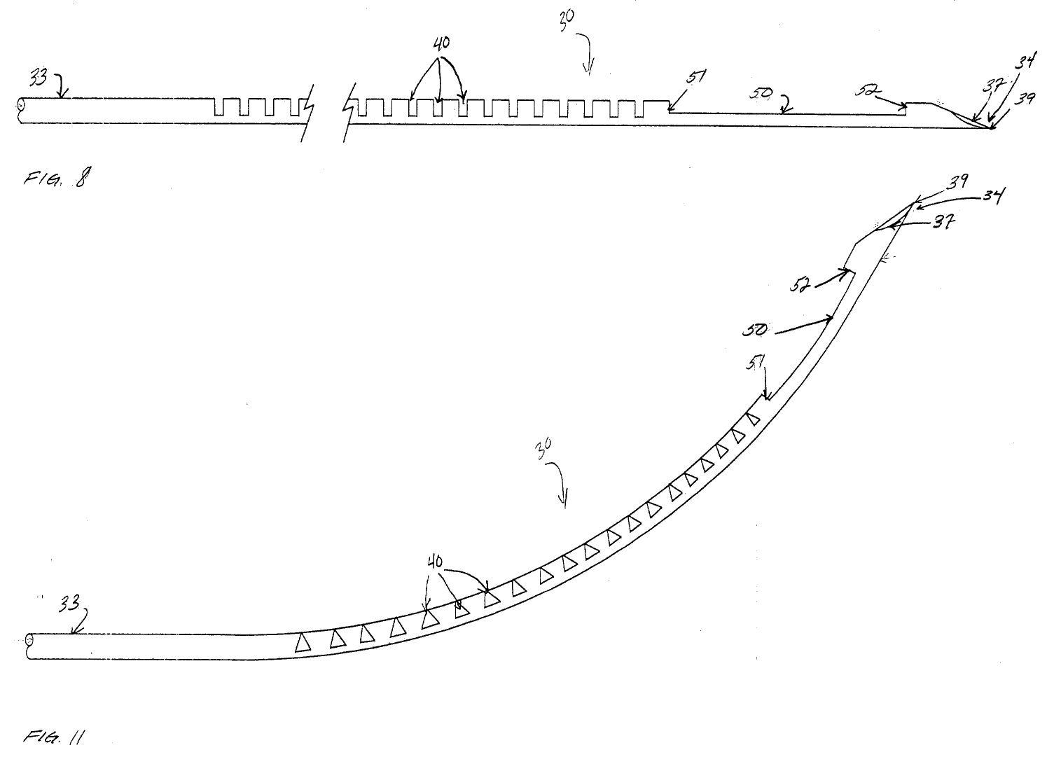

The parts of the flexible surgical cutting instrument 5 of the present invention are arranged so that the at least one cannula and the at least one stylet notches 20, 40 are facing the same direction, which also defines the direction of the bend or curve of the flexible surgical cutting instrument 5. In the preferred embodiment, the at least one cannula notch 20 at least partly overlaps the at least one stylet notch 40 when the cannula 10 is advanced over the stylet 30. Several preferred embodiments have been contemplated. As shown in FIGS. 7 and 10, a plurality of cannula notches 20 may at least partly overlap a plurality of stylet notches 40. Alternatively, as shown in FIGS. 13 and 16, a single cannula notch 20 may at least partly overlap a single stylet notch 40. Alternatively, a single cannula 20 may at least partly overlap a plurality of stylet notches 40 and, conversely, a plurality of cannula notches 40 may at least partly overlap a single stylet notch 20. Preferably, the cannula forward shearing point 19 is angularly disposed 180° from the stylet forward piercing point 39 as shown in FIGS. 1, 7, 10, 13 and 16.

In operation, the flexible surgical cutting instrument 5 of the present invention may be employed to negotiate a curve as it is manipulated through the vasculature of a patient and still retain the ability for the cannula 10 to move smoothly and freely over stylet 30 to biopsy a tissue specimen. The flexible surgical cutting instrument 5 of the present invention may be inserted through an outer or guiding catheter that is located in the biliary tree, for example. The present invention enables the physician to biopsy a target lesion directly from within the biliary tree and without traversing healthy tissue. The cannula and the stylet notches 20, 40 permit the cannula 10 and the stylet 30, respectively, to bend in a plane perpendicular to the plane of the notch and prevents the cannula 10 from binding as it is advanced over stylet 30. In addition, the distance along which the at least one cannula and the at least one stylet notches 20, 40 are positioned along the cannula wall 18 and the stylet surface 38, respectively, controls the radius of curvature that results.

It is to be understood that the above-described flexible surgical cutting instrument is merely an illustrative embodiment of the principles of this invention and that other cutting instruments may be devised by those skilled in the art without departing from the spirit and scope of this invention. In particular, the distal end of the cannula 10 may be devised to include serrated teeth or a modified cutting edge for providing any number of different cutting or slicing actions. The distal end 34 of stylet 30 may be devised to achieve any number of different piercing actions. In another embodiment, only the stylet is “notched,” i.e., the stylet comprises at least one stylet notch 40, whereas the cannula is formed from a flexible material without a cannula notch 20. Alternatively, in yet another embodiment, only the cannula is notched, i.e. the cannula comprises at least one notch the cannula, whereas the stylet is formed from a flexible material without a stylet notch.

1. A flexible surgical cutting instrument comprising:
a cannula comprising a cannula wall, a cannula proximal end, a cannula distal end, a cannula proximal portion, a cannula distal portion and at least one cannula notch; and
a stylet comprising a stylet wall, a stylet proximal end, a stylet distal end, a stylet proximal portion, a stylet distal portion and at least one stylet notch, the stylet slidably disposed within the cannula;
wherein the at least one cannula notch at least partially overlaps the at least one stylet notch.

2. The flexible surgical cutting instrument of claim 1, wherein the at least one cannula notch is positioned along the cannula wall proximal the cannula distal end.

3. The flexible surgical cutting instrument of claim 1, wherein the at least one cannula notch is positioned along the cannula wall in the cannula distal portion.

4. The flexible surgical cutting instrument of claim 1, wherein the at least one cannula notch is positioned along the cannula wall over a distance between the cannula proximal end and the cannula distal end.

5. The flexible surgical cutting instrument of claim 1, wherein the at least one cannula notch is positioned along the cannula wall over a distance from about 5 cm to about 10 cm.

6. The flexible surgical cutting instrument of claim 1, wherein the cannula wall further comprises a cannula wall periphery and the at least one cannula notch reduces the cannula wall periphery by at least one half.

7. The flexible surgical cutting instrument of claim 1, wherein the cannula distal end further comprises a distal shearing end.

8. The flexible surgical cutting instrument of claim 7, wherein the distal shearing end further comprises a shearing edge and a cannula shearing point.

9. The flexible surgical cutting instrument of claim 1, wherein the at least one stylet notch is positioned along the stylet wall proximal the stylet distal end.

10. The flexible surgical cutting instrument of claim 1, wherein the at least one stylet notch is positioned along the stylet wall in the stylet distal portion.

11. The flexible surgical cutting instrument of claim 1, wherein the at least one stylet notch is positioned along the stylet wall over a distance between the stylet proximal end and the stylet distal end.

12. The flexible surgical cutting instrument of claim 1, wherein the at least one stylet notch is positioned along the stylet wall over a distance from about 5 cm to about 10 cm.

13. The flexible surgical cutting instrument of claim 1, wherein the stylet wall further comprises a stylet wall periphery and the at least one stylet notch reduces the stylet wall periphery by at least one half.

14. The flexible surgical cutting instrument of claim 1, wherein the stylet further comprises a specimen notch positioned proximal the stylet distal end.

15. The flexible surgical cutting instrument of claim 14, wherein the at least one stylet notch is positioned proximal the specimen notch.

16. The flexible surgical cutting instrument of claim 8, wherein the stylet distal end further comprises a piercing end, the piercing end comprising a cutting edge and a stylet piercing point.

17. The flexible surgical cutting instrument of claim 16, wherein the stylet piercing point is angularly disposed about 180 degrees from the cannula shearing point.

18. The flexible surgical cutting instrument of claim 1, further comprising a handle connected to the cannula and the stylet for providing relative movement between the cannula and the stylet from a first position to a second position;
wherein in the first position, the stylet is at least partially retracted within the cannula; and
wherein in the second position, the stylet at least partially extends beyond the cannula.

19. The flexible surgical cutting instrument of claim 18, wherein the handle is a spring-loaded handle.

20. A surgical cutting instrument comprising:
a cannula comprising a cannula wall, a cannula proximal end, a cannula distal end, a cannula proximal portion, a cannula distal portion and a plurality of cannula notches positioned along the cannula wall proximal the cannula distal end over a distance from between the cannula proximal end and the cannula distal end,
the cannula wall further comprising a cannula wall periphery and the at least one cannula notch reduces the cannula wall periphery by about one half;
a stylet comprising a stylet surface, a stylet proximal end, a stylet distal end, a stylet proximal portion, a stylet distal portion, a specimen notch and a plurality of stylet notches,
the specimen notch positioned along the stylet surface proximal the stylet distal end,
the plurality of stylet notches positioned along the stylet surface proximal the specimen notch over a distance between the stylet proximal end and the specimen notch,
the stylet surface further comprising a stylet surface periphery and each of the plurality of stylet notches reduces the stylet surface periphery by at least one half; and
a spring-loaded handle mechanism to advance the cannula over the stylet;
wherein the stylet is slidably disposed within the cannula such that the plurality of cannula notches at least partially overlaps the plurality of stylet notches.

21. A surgical cutting instrument comprising:
a cannula comprising a cannula wall, a cannula proximal end, a cannula distal end, a cannula proximal portion, a cannula distal portion and at least one cannula notch positioned along the cannula wall proximal the cannula distal end over a distance from between the cannula proximal end and the cannula distal end,
the cannula wall further comprising a cannula wall periphery and the at least one cannula notch reduces the cannula wall periphery by about one half,
the cannula distal end further comprising a shearing edge and a cannula shearing point;
a stylet comprising a stylet surface, a stylet proximal end, a stylet distal end, a stylet proximal portion, a stylet distal portion, a specimen notch and at least one stylet notch,
the specimen notch positioned along the stylet surface proximal the stylet distal end,
the at least one stylet notch positioned along the stylet surface proximal the specimen notch over a distance between the stylet proximal end and the specimen notch,
the stylet surface further comprising a stylet surface periphery and the at least one stylet notch reduces the stylet surface periphery by at least one half;
the stylet distal end further comprising a cutting edge and a stylet piercing point; and
a spring-loaded handle mechanism to advance the cannula over the stylet;
wherein the stylet is slidably disposed within the cannula such that the at least one cannula notch at least partially overlaps the at east one stylet notch; and
wherein the stylet piercing point is angularly disposed about 180 degrees from the cannula shearing point.
